(19) United States
(12) Patent Application Publication
(10) Pub. No.: US 2024/0398733 A1
(43) Pub. Date: Dec. 5, 2024

(54) METHOD OF ADMINISTERING OXYBATE

(71) Applicant: Jazz Pharmaceuticals Ireland Limited, Dublin (IE)

(72) Inventor: Franck SKOBIERANDA, Palo Alto, CA (US)

(21) Appl. No.: 18/699,363

(22) PCT Filed: Oct. 11, 2022
(86) PCT No.: PCT/EP2022/078269

§ 371 Date: Apr. 8, 2024

Related U.S. Application Data
(60) Provisional application No. 63/254,270, filed on Oct. 11, 2021.

(51) Int. Cl. A61K 31/19

(52) U.S. Cl. CPC A61K 31/19

(57) ABSTRACT

Provided herein are methods of administering different GHB forms on different days for the treatment of narcolepsy and other conditions.

### CROSS-REFERENCE TO RELATED APPLICATIONS

This application claims priority to U.S. Application. No. 63/254,270, filed on Oct. 11, 2021. The contents of the aforementioned application are incorporated herein by reference in its entirety.

### BACKGROUND

Gamma-hydroxybutyrate (GHB), also known as “oxybate,” is an endogenous compound with hypnotic properties that is found in many human body tissues. GHB is present, for example, in the mammalian brain and other tissues. In the brain, the highest GHB concentration is found in the hypothalamus and basal ganglia and GHB is postulated to function as a neurotransmitter (Snead and Morley, 1981, Brain Res. 227 (4): 579-89). The neuropharmacologic effects of GHB include increases in brain acetylcholine, increases in brain dopamine, inhibition of GABA-ketoglutarate transaminase and depression of glucose utilization but not oxygen consumption in the brain. GHB treatment substantially reduces the signs and symptoms of narcolepsy, i.e., daytime sleepiness, cataplexy, sleep paralysis, and hypnagogic hallucinations. In addition, GHB increases total sleep time and REM sleep, and it decreases REM latency, reduces sleep apnea, and improves general anesthesia (e.g., U.S. Pat. Nos. 6,472,431; 6,780,889; 7,262,219; 7,851,506; 8,263,650; and 8,324,275, the disclosure of each of which is incorporated by reference in its entirety for all purposes). GHB has been formulated in a variety of ways.

Sodium oxybate (Na.GHB), commercially sold as Xyrem®, is an FDA approved formulation for the treatment of excessive daytime sleepiness and cataplexy in patients with narcolepsy. Na.GHB has also been reported to be effective for relieving pain and improving function in patients with fibromyalgia syndrome (See Scharf et al., 2003, J. Rheumatol. 30:1070; Russell et al., 2009, Arthritis. Rheum. 60:299), in treating alcohol addiction and alcohol withdrawal syndrome (See Keating, G M, 2014 January; 34 (1): 63-80), in alleviating excessive daytime sleepiness and fatigue in patients with Parkinson's disease, improving myoclonus and essential tremor, and reducing tardive dyskinesia and bipolar disorder (See Ondo et al., 2008, Arch. Neural. 65:1337; Frucht et al., 2005, Neurology 65:1967; Berner, 2008, J. Clin. Psychiatry 69:

862). There is another FDA approved formulation of GHB which contains less sodium. See Xywav® which is described below.

There is a need to develop new methods of administration of GHB forms.

### SUMMARY

In one aspect, the present disclosure provides a method for treating a GHB-treatable disorder comprising: administering at least two forms of GHB to a patient in need thereof, wherein the administration of each form provides a different pharmacokinetic profile and each form is administered on different days.

In some embodiments, the present invention is a method for treating a GHB-treatable disorder comprising administering at least two forms of GHB having different GHB release rates, to a patient in need thereof, wherein on one day, or series of days, an immediate release form is administered to the patient; and wherein a modified release form is administered to the patient on other days. In some embodiments, the immediate release form is administered more than once per night. In some embodiments, the immediate release form is administered twice per night. In some embodiments, the immediate release form is administered three times per night. In some embodiments, the modified release form is administered once per night.

In some embodiments, the present invention provides a drug distribution system for providing at least two forms of GHB to a patient to treat a sleep related disorder, comprising instructing the patient to administer the two forms within a dosing period, wherein the patient is instructed to administer the two forms on different days in the dosing period. In some embodiments, the GHB formulation is distributed using a system having a central database and a single pharmacy.

In some embodiments, the GHB-treatable disorders are one or more conditions selected from the group consisting of narcolepsy, cataplexy, excessive daytime sleepiness, idiopathic hypersomnia, cataplexy, sleep paralysis, apnea, narcolepsy sleep time disturbances, hypnagogic hallucinations, sleep arousal, insomnia, and nocturnal myoclonus or disorders related to drug abuse, alcohol and opiate withdrawal, a reduced level of growth hormone, anxiety, analgesia, effects in certain neurological disorders such as Parkinson's Disease, depression, certain endocrine disturbances and tissue protection following hypoxia/anoxia such as in stroke or myocardial infarction, or for an increased level of intracranial pressure or the like.

In some embodiments, the present invention provides a packaged product containing GHB unit doses, where the unit doses comprise at least two forms of GHB having different release rates. In some embodiments, the packaged product comprises unit dose forms selected from blister packs or sachets.

In some embodiments, of the present invention, a health care provider co-prescribes a custom dosing regimen or a preset dosing regimen, having a defined dosing period, under conditions in which one form is administered to achieve one clinical effect and another form is administered to achieve a different clinical effect.

In some embodiments, the GHB is present in a formulation having two or more cations. In some embodiments, the GHB is present in a formulation having two or more cations selected from the group consisting of sodium, potassium, magnesium and calcium. In some embodiments, the formulation has less than 50%, 40%, 30%, 20%, 10% sodium as a cation, preferably 8% sodium.

In some embodiments, the GHB form administered is selected to produce heightened alertness, to modify the administration of GHB, including the form and the number of nightly doses, to increase the intended effect and/or reduce unintended effects for a given patient; i.e., optimize/customize/adapt the administration.

In some embodiments, the GHB is administered in different forms on different days, such as one form on the nights preceding workdays and a different form on nights preceding non-workdays. In some embodiments, a twice nightly form is administered on Sunday through Thursday nights and a once nightly form is administered on Friday and Saturday nights.

In some embodiments, the GHB is administered at a reduced dosage when co-administered with sodium valproate.

In some embodiments, the administration of an immediate release form provides heightened alertness in the wake period following administration compared to the wake period following administration of a sustained release form.

The administration could also be to shift an effect, such as a shift of an effect to a different part of a sleep period to increase a therapeutic effect or reduce an unintended effect. The present invention is also a drug distribution system in which at least two forms of a GHB formulation are provided to a patient to treat a sleep related disorder, comprising co-prescribing the two forms to be delivered within the same dosing period, the two forms are to be administered on different days in the same dosing period. The two forms include an immediate release and a modified release form. In some embodiments, the invention is a method for treating a GHB-treatable disorder, comprising administering at least two forms of GHB on different days, but within 10 days of each other, to a patient in need thereof. A first form is administered to the patient to increase, reduce, or shift the intended effect for a given patient; i.e., optimize, customize, or adapt the administration to effect the patient in the wake period following administration of the first form. The second form is administered to the patient to increase, reduce, or shift the unintended effects for a given patient in the wake period following administration of the second form; wherein the two administrations are provided on different days.

The invention also includes a method wherein a health care provider co-prescribes a custom dosing regimen or a preset dosing regimen, having a defined dosing period, under conditions in which one form is given (e.g. a day where wakefulness is at a premium, such as a work day) versus the conditions under which another form is given (e.g. a day where wakefulness is not critical, such as a non-work day).

### Definitions

The following patents, publications and application are related to the present disclosure and are hereby incorporated by reference in their entireties for all purposes: U.S. Pat. Nos. 6,472,431; 6,780,889; 7,262,219; 8,263,650; 8,461,203; 8,859,619; 9,539,330; 7,851,506; 8,324,275; 8,952,062; 8,731,963; 8,772,306; 8,952,029; 9,050,302; 9,486,426; 10,213,400; 8,591,922; 8,901,173; 9,132,107; 9,555,017; 10,195,168; 8,778,301; 9,801,852; 8,771,735; 8,778,398; 9,795,567; 9,801,852, 10,398,662, 10,758,488, 10,813,885, U.S. Patent Publication Nos. US 2018/0042855 and 2018/0263936, and U.S. application Ser. Nos. 16/688,797, 17/130,769, 17/131,418, 17/180,991, 62/769,380, 62/769,382, 63/092,833, and 63/142,738.

Throughout this disclosure, various patents, patent applications and publications are referenced. The disclosures of these patents, patent applications and publications in their entireties are incorporated into this disclosure by reference for all purposes in order to more fully describe the state of the art as known to those skilled therein as of the date of this disclosure. This disclosure will govern in the instance that there is any inconsistency between the patents, patent applications and publications cited and this disclosure.

For convenience, certain terms employed in the specification, examples and claims are collected here. Unless defined otherwise, all technical and scientific terms used in this disclosure have the same meanings as commonly understood by one of ordinary skill in the art to which this disclosure belongs.

The term “about” when immediately preceding a numerical value means a range (e.g., plus or minus 10% of that value). For example, “about 50” can mean 45 to 55, “about 25,000” can mean 22,500 to 27,500, etc., unless the context of the disclosure indicates otherwise, or is inconsistent with such an interpretation. For example, in a list of numerical values such as “about 49, about 50, about 55, . . . ”, “about 50” means a range extending to less than half the interval(s) between the preceding and subsequent values, e.g., more than 49.5 to less than 52.5. Furthermore, the phrases “less than about” a value or “greater than about” a value should be understood in view of the definition of the term “about” provided herein. Similarly, the term “about” when preceding a series of numerical values or a range of values (e.g., “about 10, 20, 30” or “about 10-30”) refers, respectively to all values in the series, or the endpoints of the range.

The terms “administer,” “administering” or “administration” as used herein refer to directly administering a compound or pharmaceutically acceptable salt of the compound or a composition or formulation comprising the compound or pharmaceutically acceptable salt of the compound to a patient.

As used herein, the term “gamma-hydroxybutyrate” (GHB) or “oxybate” refers to the negatively charged or anionic form (conjugate base) of gamma-hydroxybutyric acid. GHB has the following structural formula:

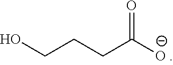

As used herein, the term “gamma-hydroxybutyric acid” (GBA) refers to the protonated form (conjugate acid) of gamma-hydroxybutyrate. GBA has the following structural formula:

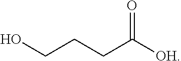

Salt forms of GHB are disclosed in U.S. Pat. Nos. 8,591,922; 8,901,173; 9,132,107; 9,555,017; and 10,195,168, which are hereby incorporated by reference in their entireties for all purposes.

The terms “effective amount” and “therapeutically effective amount” are used interchangeably in this disclosure and refer to an amount of a compound, or a salt thereof, that, when administered to a patient, is capable of performing the intended result. For example, an effective amount of a mixed salt oxybate is that amount which is required to reduce cataplexy in a patient. The actual amount which comprises the “effective amount” or “therapeutically effective amount” will vary depending on a number of conditions including, but not limited to, the severity of the disorder, the size and health of the patient, and the route of administration. A skilled medical practitioner can readily determine the appropriate amount using methods known in the medical arts.

As used herein, the term “form” when applied to “GHB” means a version of GHB that has a property that is different from other forms of GHB. For example, forms may differ by function, such as release rate (for example immediate, modified, sustained, controlled and other releases, see U.S. Pat. No. 10,758,488 and U.S. application Ser. No. 16/688,797), physical state (liquid, solid, including particle formation or other solid forms), formulations, or by manufacturer (branded or generic). Example GHB forms include Xyrem (sodium oxybate, see U.S. Pat. No. 6,472,431), Xywav (mixed salt oxybate, see U.S. Pat. No. 8,591,922), GHB particles (See U.S. Pat. No. 10,398,662), GHB polymers (See U.S. Pat. No. 8,778,301), and GBL. Other forms that have different GHB release characteristics are also included.

As used herein, the term “equivalent”, when comparing Na. GHB and mixed salt oxybate forms, means both forms contain the same amount of GHB within about 5% (by weight % or % molar equivalent). In preferred embodiments, a liquid formulation of a mixed salt oxybate is equivalent to the Na.GHB-containing liquid formulation Xyrem (which contains 0.409 g/mL of GHB).

In some embodiments, a liquid formulation of a mixed salt contains 0.234 g/mL of calcium oxybate, 0.130 g/mL of potassium oxybate, 0.096 g/mL of magnesium oxybate, and 0.040 g/mL of sodium oxybate.

As used herein, the term “patient” refers to a mammal, particularly a human.

The phrase “pharmaceutically acceptable” as used herein refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, “carrier” encompasses solvents, dispersion media, coatings, antibacterial and antifungal agents, isotonic and absorption delaying agents and the like. The use of carriers for active pharmaceutical ingredients is well known in the art. Insofar as any conventional media or agent is incompatible with the active ingredient, its use in the therapeutic compositions is not appropriate.

The term “therapeutic effect” as used herein refers to a desired or beneficial effect provided by the method and/or the composition. For example, the method for treating cataplexy provides a therapeutic effect when the method reduces cataplexy.

The term “treating” as used herein with regard to a patient, refers to improving at least one symptom of the patient's disorder. Treating can be curing, improving, or at least partially ameliorating a disorder.

The terms “substitute”, “switch”, “change” and “exchange” are used interchangeably in the context of the present disclosure. The methods of the present disclosure may also be expressed in terms of “transitioning from” sodium oxybate to a mixed salt oxybate.

The term “salt” or “salts,” as used herein, refers to a compound formed by the interaction of an acid and a base, the hydrogen atoms of the acid being replaced by the positive ion or cation of the base. Pharmaceutically acceptable salts include inorganic acids such as, for example, hydrochloric or phosphoric acids, or such organic acids as malic, acetic, oxalic, tartaric, mandelic, and the like. Salts formed can also be derived from inorganic bases such as, for example, sodium, potassium, silicates, ammonium, calcium, or ferric hydroxides, and such organic bases as isopropylamine, trimethylamine, histidine, procaine and the like. In certain preferred embodiments, the salt is formed from an inorganic base that is a metal, for example, an alkali metal, such as lithium, potassium, sodium, or the like, an alkaline earth metal, such as magnesium, calcium, barium, or the like, or aluminum or zinc. Other salts may comprise ammonium. Alkali metals, such as lithium, potassium, sodium, and the like, may be used, preferably with an acid to form a pH adjusting agent. Examples of pharmaceutically acceptable base addition salts include those derived from inorganic bases like sodium hydroxide, potassium hydroxide, magnesium hydroxide, calcium hydroxide, or ammonium hydroxide, and the like (See, e.g., Berge et al., 1977, J. Pharm. Sci. 66:1).

As used herein, the terms “salt of GHB” or “salts of GHB,” as used herein, refer to a compound formed by the interaction of gamma-hydroxybutyric acid (the conjugate acid of GHB) with a base, for example, NaOH, KOH, Mg(OH)₂, and Ca(OH)₂, and the like, the hydrogen atoms of the acid being replaced by the positive ion or cation of the base. Such salts may include, for example, sodium oxybate (“Na.GHB”), potassium oxybate (“K.GHB”), magnesium oxybate (“Mg.(GHB)₂”), and calcium oxybate (“Ca.(GHB)₂”), and the like. It will be understood by those skilled in the art that such salts may be in solid form, or such salts may be in partially or fully solvated form, for example, as when dissolved in an aqueous medium. It will be further understood by those skilled in the art, that, depending on the solubility of the salt in the aqueous medium, that the salt may be present in the aqueous medium as solvated cation(s) and anion(s), or as a precipitated solid.

The term “oxybate dosing strength” refers to the amount of GHB in a particular dose (e.g., each mL of Xyrem contains 0.5 g of sodium oxybate, which is equivalent to a 0.409 g/mL oxybate dosing strength). Although throughout the present disclosure, the oxybate dosing strength in a composition is generally expressed in terms of the amount of oxybate present in a composition, the present disclosure contemplates embodiments where the oxybate dosing strength is expressed in the Equivalent Concentration of GBA that is contained in the dose.

The Equivalent Concentration of GBA in a compositions may be calculated by the following formula:

$$Equivalent Concentration of GBA = \frac{\begin{matrix}Concentration of GHB in ( g / mL ) \times \\ 104.1 ( Formula Weight of GBA , \frac{g}{mol} )\end{matrix}}{103.1 ( Formula Weight of GHB ( \frac{g}{mol} )}$$

Thus, each mL of Xyrem contains 0.5 g of sodium oxybate, which is equivalent to an Equivalent Concentration of GBA of 0.413 g/mL.

The term “mixed salts” or “mixed salt oxybate,” as used herein, refers to salts of GHB where two, three, four or more different cations are present in combination with each other in a composition. Such mixtures of salts may include, for example, salts selected from the group consisting of Na.GHB, K.GHB, Mg. (GHB) 2, and Ca. (GHB) 2. Mixed salt oxybates are described in U.S. Pat. Nos. 8,591,922; 8,901,173; 9,132,107; 9,555,017; and 10,195,168, the contents of which is hereby incorporated by reference it entirety for all purposes.

The term “JZP-258” as used herein refers to a solution containing the mixed salt oxybate comprising about 8% sodium oxybate, about 23% potassium oxybate, about 21% magnesium oxybate and about 48% calcium oxybate (% mol. equiv. of GHB) and having a GHB concentration of 0.409 g/mL (or, expressed another way, an Equivalent Concentration of GBA of 0.413 g/mL). The following table describes the % mol. equiv., wt/vol %, and absolute amount of sodium oxybate, potassium oxybate, magnesium oxybate and calcium oxybate in representative doses of JZP-258.

| | % mol | wt/ | Amount in 1 g | | Amount in 9 g | |
|---|---|---|---|---|---|---|
| | equivalent | wt % | JZP-258 | | JZP-258 | |
| Na•GHB | 8 | 8 | 80 | mg | 720 | mg |
| K•GHB | 23 | 25.5 | 255 | mg | 2,295 | mg |
| Mg•(GHB)₂ | 21 | 19.5 | 195 | mg | 1,755 | mg |
| Ca•(GHB)₂ | 48 | 47 | 470 | mg | 4,230 | mg |

The term “wt/wt %,” as used herein, refers to the normalized weight percent of a particular salt in a salt mixture.

The term “wt/wt % ratio,” as used herein, refers to the ratio of wt/wt % values in a mixture of salt. For example, where the salts Na. GHB, K.GHB, Mg. (GHB) 2, and Ca. (GHB) 2 are present in a wt/wt % of 8%, 25.5%, 19.5% and 47%, respectively, the wt/wt % ratio of Na.GHB, K.GHB, Mg. (GHB) 2, and Ca. (GHB) 2 in the mixture is 8:25.5:19.5:47.

The term “wt/vol %,” as used herein, refers to the normalized weight percent of a particular salt in a particular volume of solution.

The term, “formulation,” as used herein, refers to a stable and pharmaceutically acceptable preparation of a pharmaceutical composition disclosed herein.

The term, “liquid formulation,” as used herein, refers to a water-based formulation, in particular, a formulation that is an aqueous solution.

### DETAILED DESCRIPTION

The present invention involves prescribing different forms of oxybate, also known as gamma hydroxybutyrate (GHB). Preferred forms include sodium oxybate, a mixed salt form of oxybate, and combinations thereof which have different release profiles. For example, forms that have a modified release profile.

Sodium oxybate (Na.GHB), commercially sold as Xyrem®, is approved for the treatment of cataplexy or excessive daytime sleepiness in patients 7 years of age or older with narcolepsy. The approved daily dose of Xyrem® is 6-9 grams per night administered orally. See U.S. Pat. Nos. 6,472,431 and 8,731,963 which are incorporated by reference in their entireties.

Xywav® was developed to provide the same treatment benefits as Xyrem with substantially less sodium, so that patients with the lifelong disease of narcolepsy could be more able to achieve daily sodium intake goals for optimum health. See U.S. Pat. Nos. 8,591,922 and 10,675,258 which are incorporated by reference in their entireties.

Xywav® is a mixed salt oxybate that contains calcium oxybate, magnesium oxybate, potassium oxybate, and sodium oxybate, and it provides 87-131 mg of sodium when administered in the dose range of 6-9 grams nightly. This amount is 92% less sodium than that provided by Xyrem® administration of an equivalent dose. Though important for every person, daily sodium intake goals are a vital consideration for all patients with the lifelong disease of narcolepsy, given the increased presence of multiple cardiovascular comorbidities, including hypertension, congestive heart failure, and myocardial infarction (Jennum P, et al. Comorbidity and mortality of narcolepsy: a controlled retro- and prospective national study. Sleep. 2013 Jun. 1; 36 (6): 835-40.; Ohayon MM. Narcolepsy is complicated by high medical and psychiatric comorbidities: a comparison with the general population. Sleep Med. 2013 June; 14 (6): 488-92.; and Black J, et al. Medical comorbidity in narcolepsy: findings from the Burden of Narcolepsy Disease (BOND) study. Sleep Med. 2017 May; 33:13-18.).

### Mixed Salt Oxybate

In some embodiments, the methods of the present disclosure comprise administering a mixed salt oxybate to a patient in need thereof.

In some embodiments, the mixed salt oxybate comprises gamma-hydroxybutyrate (GHB) and three or four or more pharmaceutically acceptable cations of an alkali metal or an alkaline earth metal.

In some embodiments, the mixed salt oxybate comprises GHB and more than one pharmaceutically acceptable cations of an alkali metal or an alkaline earth metal.

In some embodiments, the mixed salt oxybate comprises GHB and two, three, or four cations selected from the group consisting of Na, K⁺, Mg⁺², and Ca⁺². In some embodiments, mixed salt oxybate comprises GHB and all three cations selected from the group consisting of K⁺, Mg⁺², and Ca⁺². In some embodiments, the mixed salt oxybate does not contain Na⁺, or comprises less of, Na⁺.

In some embodiments, the mixed salt oxybate comprises two, three, or four salts selected from the group consisting of a sodium salt of hydroxybutyrate (Na.GHB), a potassium salt of gamma-hydroxybutyrate (K.GHB), a magnesium salt of gamma-hydroxybutyrate (Mg.(GHB)₂), and a calcium salt of gamma-hydroxybutyrate (Ca.(GHB)₂).

In some embodiments, the mixed salt oxybate comprises varying weight/weight percentages (wt/wt %) of Na.GHB, K.GHB, Mg.(GHB)₂, and Ca.(GHB)₂.

In some embodiments, any of the salts, such as the Na.GHB salt, the K.GHB salt, the Mg.(GHB)₂ salt or the Ca.(GHB)₂, is present in about 1%-5%, about 5%-10%, about 10%-15%, about 15%-20%, about 20%-25%, about 25%-30%, about 30%-35%, about 35%-40%, about 40%-45%, about 45%-50%, about 50%-55%, about 55%-60%, about 60%-65%, about 65%-70%, about 70%-75%, about 75%-80%, about 80%-85%, about 85%-90%, about 90%-95%, or about 95%-100% (wt/wt %). In some embodiments, the Na.GHB salt is present in a wt/wt % of about 1%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% (wt/wt %). In some embodiments, the Na. GHB salt is absent.

In some embodiments, where the mixed salt oxybate comprises a mixture of Na. GHB, K.GHB, Mg. (GHB) 2, and Ca. (GHB) 2, the Na.GHB salt is present in a wt/wt % of about 1%-15%, 5%-10%, or about 8%; the K.GHB salt is present in a wt/wt % of about 10%-30%, 15%-25%, or about 25.5%; the Mg. (GHB) 2 salt is present in a wt/wt % of about 10%-30%, 15%-25%, or about 19.5%; and the Ca. (GHB) 2 salt is present in a wt/wt % of about 30%-60%, 40%-50, or about 47% (wt/wt %).

In some embodiments, the mixed salt oxybate comprises about 8% of sodium oxybate (wt/wt %), about 25.5% of potassium oxybate (wt/wt %), about 19.5% of magnesium oxybate (wt/wt %) and about 47% of calcium oxybate (wt/wt %). In some embodiments, where the mixed salt oxybate comprises a mixture of Na.GHB, K.GHB, Mg. (GHB) 2, and Ca. (GHB) 2, the Na.GHB, K.GHB, Mg. (GHB) 2, and Ca. (GHB) 2 salts are present in a wt/wt % ratio of about 8:25.5:19.5:47, respectively.

In some embodiments, a mixed salt oxybate of the present disclosure is dissolved in a liquid (such as water) to provide a pharmaceutical composition and the concentration of the mixed salt oxybate is expressed in terms of the wt/vol %. In some embodiments, where the mixed salt oxybate comprises a mixture of Na.GHB, K.GHB, Mg. (GHB) 2, and Ca. (GHB) 2, the Na.GHB salt is present in a wt/vol % of about 1%-15%, 5%-10%, or about 8%; the K.GHB salt is present in a wt/vol % of about 10%-30%, 15%-25%, or about 26%; the Mg. (GHB) 2 salt is present in a wt/vol % of about 10%-30%, 15%-25%, or about 19.2%; and the Ca. (GHB) 2 salt is present in a wt/vol % of about 30%-60%, 40%-50, or about 46.8% (wt/vol %).

In some embodiments, the liquid pharmaceutical composition containing the mixed salt oxybate comprises about 8% of sodium oxybate (wt/vol %), about 23.0% of potassium oxybate (wt/vol %), about 21% of magnesium oxybate (wt/vol %) and about 48% of calcium oxybate (wt/vol %).

In some embodiments, the mixed salt oxybate comprises varying percentages of oxybate, expressed as % molar equivalents (% mol. equiv.) of Na.GHB, K.GHB, Mg. (GHB) 2, and Ca. (GHB) 2. The terms “% molar equivalents” and “% mol. equiv.,” as used herein, refer to molar composition of salts expressed as a percent of GHB equivalents. Those skilled in the art will understand that as each GHB unit is considered to be one molar equivalent, the monovalent cations, Na′ and K′, have one molar equivalent per salt, and the divalent cations, Mg*2 and Ca+2, have two molar equivalents per salt. See U.S. Pat. Nos. 8,591,922; 8,901,173; 9,132,107; 9,555,017; 10,195,168 for amounts of % mol. equiv. useful in the present disclosure.

In some embodiments, any of the salts, such as the Na. GHB salt, the K.GHB salt, the Mg. (GHB) 2 salt or the Ca. (GHB) 2, is present in about 1%-5%, about 5%-10%, about 10%-15%, about 15%-20%, about 20%-25%, about 25%-30%, about 30%-35%, about 35%-40%, about 40%-45%, about 45%-50%, about 50%-55%, about 55%-60%, about 60%-65%, about 65%-70%, about 70%-75%, about 75%-80%, about 80%-85%, about 85%-90%, about 90%-95%, or about 95%-100% (% mol. equiv.). In some embodiments, the Na.GHB salt is present in a % mol. equiv. of about 1%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% (% mol. equiv.). In some embodiments, the Na. GHB salt is absent.

In some embodiments, where the mixed salt oxybate comprises a mixture of Na.GHB, K.GHB, Mg. (GHB) 2, and Ca. (GHB) 2, the Na.GHB salt is present in a % mol. equiv. of about 1%-15%, 5%-10%, or about 8%; the K.GHB salt is present in a % mol. equiv. of about 10%-30%, 15%-25%, or about 23%; the Mg. (GHB) 2 salt is present in a % mol. equiv. of about 10%-30%, 15%-25%, or about 21%; and the Ca. (GHB) 2 salt is present in a % mol. equiv. of about 30%-60%, 40%-50, or about 48% (% mol. equiv.).

In some embodiments, the mixed salt oxybate comprises about 8% mol. equiv. of sodium oxybate, about 23% mol. equiv. of potassium oxybate, about 21% mol. equiv. of magnesium oxybate and about 48% mol. equiv. of calcium oxybate. In some embodiments, where the mixed salt oxybate comprises a mixture of Na.GHB, K.GHB, Mg. (GHB) 2, and Ca. (GHB) 2, wherein the mixture comprises Na.GHB, K.GHB, Mg. (GHB) 2, and Ca. (GHB) 2 salts are present in a % mol. equiv. ratio of about 8:23:21:48, respectively.

In some embodiments, where the pharmaceutical composition comprises a mixture of Na.GHB, K.GHB, and Ca. (GHB) 2, the Na.GHB salt is present in a % mol. equiv. of about 5%-40%, the K.GHB salt is present in a % mol. equiv. of about 10%-40%, and the Ca. (GHB) 2 salt is present in a % mol. equiv. of about 20%-80%.

### Pharmaceutical Compositions:

In some embodiments, the oxybate is in the form of a pharmaceutical composition that is suitable for administration in the methods of the present disclosure. In some embodiments, the pharmaceutical composition comprises an aqueous solution. In some embodiments, the pharmaceutical composition is a solid.

In some embodiments, the concentration of the mixture of salts of GHB in the solution is about 50 mg/mL-950 mg/mL, about 250 mg/mL-750 mg/mL, about 350 mg/mL-650 mg/mL, or about 450 mg/mL-550 mg/mL. In some embodiments, the concentration of the mixture of salts of GHB in the solution is about 500 mg/mL.

In some embodiments, the pH of the pharmaceutical composition is about 7.0-9.0, about 7.0-8.5, or about 7.3-8.5.

In some embodiments, the pharmaceutical composition is chemically stable and resistant to microbial growth. In some embodiments, the pharmaceutical composition is free of preservatives. See U.S. Pat. Nos. 6,472,431; 6,780,889; 7,262,219; 8,263,650; 8,461,203 and others for a relationship between pH and GHB concentration and their effect on microbial growth.

In some embodiments, a pH adjusting or buffering agent may be added to the pharmaceutical composition. The choice of a pH adjusting or buffering agent may affect the resistance to microbial challenge and/or the stability of GHB, as measured by the reduction in assayable GHB. Pharmaceutical compositions of GHB, pH adjusted or buffered with malic acid are resistant to both microbial growth and chemical degradation of GHB and are preferred. Other pH adjusting or buffering agents may be selected. Agents that adjust pH that are selected on this basis will undergo a taste testing study. However, any pH adjusting or buffering agent disclosed herein or as would be known to those skilled in the art is contemplated as being useful from the compositions or formulations disclosed herein. Of course, any salt, flavoring agent, excipient, or other pharmaceutically acceptable addition described herein or as would be known to those skilled in the art is contemplated as being useful for the compositions or formulations disclosed herein.

In some embodiments, the pH adjusting or buffering agent is an acid. In some embodiments, the pH adjusting or buffering agent is an inorganic acid or an organic acid. In some embodiments, the pH adjusting or buffering agent is selected from the group consisting of malic acid, citric acid, acetic acid, boric acid, lactic acid, hydrochloric acid, phosphoric acid, sulfuric acid, sulfonic acid, and nitric acid. In some embodiments, the pH adjusting or buffering agent is malic acid.

The aqueous solutions disclosed herein typically comprise an effective amount of GHB, which may be dissolved or dispersed in a pharmaceutically acceptable carrier and/or an aqueous medium.

### Formulations

In some embodiments, the pharmaceutical compositions disclosed herein are provided in a formulation that is suitable for administration in the methods of the present disclosure. In some embodiments, the formulations are for oral or parenteral use. In some embodiments, the pharmaceutical compositions are formulated as dry powders, solutions, particles, suspensions, tablets, pills, capsules, modified release formulations or powders to be admixed with an aqueous medium for parenteral administration, such as intravenous or intramuscular injection, or other pharmaceutically acceptable forms include, e.g., tablets or other solids; liposomal formulations; time release capsules, such as modified or delayed release forms, including beads, pellets, or resins; and any other form currently used, including creams, which then may be admixed with an aqueous medium for oral administration. In some embodiments, oral pharmaceutical compositions comprise an inert diluent or assimilable edible carrier, or they are enclosed in hard or soft shell gelatin capsule, or are compressed into tablets, or the GHB or salt(s) thereof are packaged separately from or in combination with the excipients, salts, flavorings or any other components described herein, to be admixed with an aqueous medium for oral or injectable formulations, or they are incorporated directly with the food (i.e. a beverage) of the diet.

In some embodiments, the formulation is a liquid formulation. In some embodiments, the formulation is a solid formulation. See incorporated by reference U.S. Pat. Nos. 6,472,431; 6,780,889; 7,262,219; 8,263,650; 8,461,203, 8,591,922, 8,901,173, 9,132,107, 9,555,017, 9,795,567, 10,195,168, 10,758,488, U.S. Ser. Nos. 16/688,797, 62/769,380 and 62/769,382 and U.S. Patent Publication No. 2018/0263936 for example.

In some embodiments, the formulation is chemically stable and resistant to microbial growth. In some embodiments, the formulation free of preservatives. In some embodiments, the level of gamma-butyrolactone (GBL) is 0.1% or less of the formulation. In some embodiments, the level of gamma-butyrolactone (GBL) is 0.5% or less of the formulation.

In some embodiments, the formulation is suitable for oral administration. In some embodiments, the formulation comprises flavoring agents, sweeteners, coloring agents, surfactants, carriers, excipients, binders, or buffering compounds. See incorporated by reference U.S. Pat. Nos. 6,472,431; 6,780,889; 7,262,219; 8,263,650; 8,461,203, 8,591,922, 8,901,173, 9,132,107, 9,555,017, and 10,195,168 and U.S. Publication No. 2020/0330393 for examples of flavoring agents, sweeteners, coloring agents, surfactants, carriers, excipients, binders, buffering compounds or agents and other formulation ingredients.

In preferred embodiments, the formulation is a liquid formulation, wherein the formulation comprises 0.234 g/mL of calcium oxybate, 0.130 g/mL of potassium oxybate, 0.096 g/mL of magnesium oxybate, and 0.040 g/mL of sodium oxybate (which contains 0.409 g/mL of GHB or Equivalent Concentration of GBA of 0.413 g/mL).

In some embodiments, the formulation is immediate or modified release. In some embodiments, the formulation is suitable for administration in a single or multiple dosage regimen. See U.S. Pat. No. 10,758,488 and U.S. application Ser. No. 16/688,797.

Any of the above formulations may be prepared and/or packaged as a powdered, particle or dry form for mixing with an aqueous medium before oral administration, or they may be prepared in an aqueous medium and packaged. After mixing with an aqueous medium, preferably to prepare a solution, these formulations are resistant to both microbial growth and chemical conversion of GHB to GBL, thereby increasing the shelf-life of therapeutic formulations of GHB in an aqueous medium. These formulations then provide an easily titratable liquid medium for measuring the dosage of GHB to be administered to a patient.

The GHB may be lyophilized for more ready formulation into a desired vehicle or medium where appropriate. The active compounds may be formulated for parenteral administration, e.g., formulated for injection via intravenous, intraarterial, intramuscular, sub-cutaneous, intralesional, intraperitoneal or other parenteral routes. The preparation of a composition that comprises an aqueous solution that contains a GHB agent as an active component or ingredient will be known to those of skill in the art in light of the present disclosure. Typically, such compositions can be prepared as injectables, either as liquid solutions or suspensions. Solid forms suitable for using to prepare solutions or suspensions upon the addition of a liquid prior to injection can also be prepared; and the preparations can also be emulsified. See U.S. Pat. Nos. 6,472,431; 6,780,889; 7,262,219; 8,263,650; 8,461,203, 8,591,922, 8,901,173, 9,132,107, 9,555,017, 9,795,567, 10,195,168, U.S. Ser. No. 16/688,797 and U.S. Patent Publication No. 2018/0263936 for example for more information about parenteral administration.

Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed.

For oral therapeutic administration, the active compounds may be incorporated with excipients and used in the form of tablets, buccal tablets or tabs, troches, capsules, elixirs, suspensions, particles, syrups, wafers, and the like, to be admixed with an aqueous medium. Such compositions and preparations should contain at least 0.1% of active compound. The percentage of the compositions and preparations may, of course, be varied and may conveniently be between about 2-75% of the weight of the unit, or preferably between 25-60%. The amount of active compounds in such therapeutically useful compositions is such that a suitable dosage will be obtained. See U.S. Pat. Nos. 6,472,431; 6,780,889; 7,262,219; 8,263,650; 8,461,203, 8,591,922, 8,901,173, 9,132,107, 9,555,017, 9,795,567, 10,195,168, 10,758,488, U.S. Ser. No. 16/688,797, and U.S. Patent Publication No. 2018/0263936 for example.

The GHB form can also be a modified release form, such as a sustained release form. For example, see U.S. patent application Ser. No. 16/688,797. Immediate and modified release formulations for use in the invention, including materials and methods are shown for example in U.S. Patent Publication number 2012/0076865, and U.S. Pat. Nos. 8,771,735, 9,795,567 and 10,272,062 and 10,758,488, which are incorporated by reference in their entireties herein.

Modified release profiles are also described in (USP XXV, CDER, FDA, Rockville, Md.), extended release profiles are referenced by FDA Guidelines (“Extended Release Oral Dosage Forms: Development, Evaluation, and Application of In Vitro/In Vivo Correlations”, Food and Drug Administration, CDER, September 1997, Page 17), and immediate release profile are referenced by FDA guidelines (“Dissolution Testing of Immediate Release Solid Oral Dosage Forms”, issued August 1997, Section IV-A), all of which are incorporated herein by reference in their entireties as well as the following patents, patent applications and other cited references.

Modified release dosage forms permit the release of the active ingredient over an extended period of time in an effort to maintain therapeutically effective plasma levels over similarly extended time intervals, simplify dosing, improve dosing compliance, and/or to modify other pharmacokinetic properties of the active ingredient, such as delay onset of release or change conditions under which release occurs. Modified release formulations of the invention may provide a sustained release profile of the therapeutic agent in the absence of alcohol. Preferably, a modified release profile provides not more than about 10% to about 50% release of the therapeutic agent within about 1 hour of being in an aqueous buffer, between about 20% to about 70% release within about 2 hours to about 4 hours of being in an aqueous buffer, and between about 50% to about greater than about 80% release within about 4 hours to about 8 hours of being in an aqueous buffer.

Modified release formulations of the invention may provide a delayed release profile of the therapeutic agent in the absence of alcohol. Preferably, release of the therapeutic agent is delayed during gastric transit following ingestion. In some embodiments, release of the therapeutic agent is delayed during gastric transit following ingestion and having not more than about 0% to 40% release of the therapeutic agent within about 1 hour to about 2 hours of being in an acidic aqueous buffer (pH<5). In some embodiments, release of the therapeutic agent is delayed during exposure to the acidic aqueous buffer (pH<5), and then release of the therapeutic agent increases after the formulation is subsequently exposed to a non-acidic (pH>5) aqueous solution such that release of the therapeutic agent increases to between about 50% to about 100% release within about 1 hour of being in said non-acidic aqueous solution; or to between about 10% to about 70% release within about 1 hour to about 4 hours of being in said non-acidic aqueous solution. In the dissolution testing guideline for modified release profiles, such as those used in the present invention, material dissolves over a period of time, and its dissolution is measured at given intervals during this period. A minimum of three time points is recommended and generally cover early, middle and late stages of the dissolution profile (see Guidance for Industry, SUPAC-MR: Modified Release Solid Oral Dosage Forms,” Food and Drug Administration, CDER, September 1997). The preferred dissolution apparatus is USP apparatus I (basket) or II (paddle), used at recognized rotation speeds, e.g., 100 rpm for the basket and 50-75 rpm for the paddle.

Other modified or controlled release dissolution profiles as desirable for use in the invention are described in 16/688,797 and 10,758,488 incorporated herein.

Immediate release dosage forms are considered those that have not been engineered to modify or control the release of the active ingredient. Immediate release profiles typically provide between about 70% and about 100% release of the therapeutic agent after about 5 minutes to about 60 minutes of being in an aqueous buffer. Immediate release preferably provides dissolution profiles wherein greater than 90% of the drug included in the immediate release component is released from the immediate release component within the first hour after administration. In the dissolution testing guidelines, materials which dissolve at least 80% in the first 30 to 60 minutes in solution qualify as immediate release profiles. (“Dissolution Testing of Immediate Release Solid Oral Dosage Forms”, issued August 1997, Section IV-A). Therefore, immediate release solid oral dosage forms permit the release of most, or all, of the active ingredient over a short period of time, such as 60 minutes or less, and make rapid absorption of the drug possible. See also Patent Nos. U.S. Pat. Nos. 9,795,567, 6,472,431, 8,591,922 and 10,758,488.

A multiphase release profile (i.e., a composition containing an immediate release component and at least one modified release component) may also be employed to attain one or more combinations of release rates to attain more specific therapeutic objectives such as a portion of drug releasing immediately, followed by a modified or extended release of the remainder. See U.S. Pat. Nos. 11,065,224, 10,952,986, 10,758,488, 11,077,079, and U.S. Ser. No. 16/688,797. In some embodiments, the formulations for use in the invention comprise a drug carrier core selected from irregular particles, regular particles, spheronized particles, nanoparticles, drug-loaded non-pareils, micro-particles, pellets, beads, mini-tablets, tablets and/or capsules (hard and/or soft gelatin). In some embodiments, the drug carrier core for use in the invention comprises drug crystals. Suitable cores are described in Aulton's ‘Pharmaceutics—The Design and Manufacture of Medicines’, Chapter 32 (Aulton and Taylor; Aulton's Pharmaceutics 4th Edition, published Jun. 19, 2013) and Qiu Y., et al. ‘Developing Solid Oral Dosage Forms’, Chapters 33 and 34 (Qiu Y., et al., Developing Solid Oral Dosage Forms 1st Edition; published Dec. 19, 2008), each of which is hereby incorporated by reference in its entity for all purposes.

In some embodiments, the drug carrier core are the size and/or shape of a particle, pellet, bead, mini-tablet or tablet.

In particular embodiments, the formulations for use in the invention comprise at least two drug carrier cores each comprising a core selected from particles, nanoparticles, micro-particles, pellets, mini-tablets, tablets and/or capsules (hard and/or soft gelatin). In certain embodiments, the formulations for use in the invention comprise at least two drug carrier cores wherein at least one core comprises drug crystals.

In certain embodiments, the formulations for use in the invention are provided as a unit dosage form selected from tablets, mini-tablets, capsules, caplets, beads, pellets, particles, sachets, crystals or powders. In some embodiments, the unit dosage form may be a liquid or suspension. It is generally understood that “tablets” are meant to be those of tablet dosage forms of the art which generally are in the range of 50 mg up to 2.0 gram. Tablets can include matrix tablets, osmotic tablets, bi-layer tablets, orally disintegrating tablets, effervescent tablets and lozenges. Minitablets or mini-tablets are known in the art to be around 5 to 50 mg and typically 1 to 5 mm or preferably 1.5 to 3 mm. It is understood that drug carrier cores of the invention may comprise particles which are less than 5 mg. It is also understood that in certain embodiments, drug carrier cores of the invention comprise particles that are from 10 to 5000 microns in diameter. Pellets are multiparticulate forms that typically range from 300 to 3000 microns, from 600 to 3000 microns or preferably from 800 to 1500 microns

Solid dosage forms or drug carrier cores for use in the invention may be made by a number of techniques known in the art including, but not limited to, compression and granulation. In addition, smaller forms, such as for example, pellets (either coated or uncoated) can be compressed into a larger form, such as for example a tablet or pill of any size or shape using methods such as, for example, those described in U.S. Pat. No. 4,684,516 and Bodmeier, R. (1997) European Journal of Pharmaceutics and Biopharmaceutics, 43 (1), 1-8) which are incorporated herein by reference. Processes often used for making dosage forms of the invention also include wet granulation, dry granulation, extrusion and spheronization, hot melt extrusion, milling, sieving and blending.

An outer or external functional coating is typically applied to oral dosage forms in order to mask taste, odor or color; provide physical or chemical protection for the active ingredient/drug; control the release of the active ingredient from the formulation; protect the active ingredient from the harsh environment of the stomach (i.e. enteric coating); or protect the subject from unwanted gastrointestinal side effects. Prior to applying an external coating, a seal-coating (also referred to as a sub-coating) may first be applied. Sub-coatings can act to smooth the product surfaces, enhance the adherence of the final, outer coat, prevent migration of the drug from the core to the functional coat, and/or to protect the active ingredient from premature degradation. The present invention also shows that the outer functional coating can act to prevent or decrease alcohol-induced dose dumping. The type and/or thickness of the seal coat or the final coating(s) may be varied in order to alter product characteristics, such as dissolution. The external or functional coatings are targeted to be about 5-60% by weight (of the drug carrier core) and seal coats are targeted to be about 1-5%, preferably about 2%, by weight. Sub-coats are generally thought of as “non-functional” in that they are not utilized to control timing or placement of release of the active ingredient; however, it is considered that certain sub-coatings may act as such “functional” coatings. For the purpose of the present invention, “functional coatings” are intended to include enteric coatings, time-release coatings, pH-dependent coatings, ethanol rugged coatings, or other which control the timing or placement of release of the active ingredient. In some exemplified embodiments of the invention, the one or more separate coatings or layers of the functional coating together constitute about 40% or less, 30% or less, 20% or less, or 15% or less of the total dosage form targeted by weight. In preferred embodiments, the first functional coating is about 10-20% of the total dosage form targeted by weight. In other preferred embodiments, a second or outer functional coating is about 1-10% of the total dosage form targeted by weight.

In certain embodiments of the invention, the formulation comprises a coated drug carrier core comprising a core comprising an active agent, a first coating disposed over the core, and an optional second coating disposed over the first coating. In certain embodiments, the first coating is present at about 5%-60% w/w, inclusive of all values and subranges therebetween, including, but not limited to, 5-10%, 5-15%, 5-20%, 5-25%, 5-30%, 5-35%, 5-40%, 5-45%, 5-50%, 5-55%, 10-15%, 10-20%, 10-25%, 10-30%, 10-35%, 10-40%, 10-45%, 10-50%, 10-55%, 10-55%, 10-60, 15-20%, 15-25%, 15-30%, 15-35%, 15-40%, 15-45%, 15-50%, 15-55%, 15-60%, 20-25%, 20-30%, 20-35%, 20-40%, 20-45%, 20-50%, 20-55%, 20-60%, 25-30%, 25-35%, 25-40%, 25-45%, 25-50%, 25-55%, 25-60%, 30-35%, 30-40%, 30-45%, 30-50%, 30-55%, 30-60%, 35-40%, 35-45%, 35-50%, 35-55%, 35-60%, 40-45%, 40-50%, 40-55%, 40-60%, 45-50%, 45-55%, 45-60%, 50-55%, 50-60%, 55-60% w/w; 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 55% and 60%.

In certain embodiments of the invention, the formulation comprises a coated drug carrier core comprising a core comprising an active agent, a first coating disposed over the core, and a second coating, or “top coating” disposed over the first coating. In certain embodiments, the first coating is present at about 5%-40% w/w, inclusive of all values and subranges therebetween, and the second coating is present at about 1%-25% w/w, inclusive of all values and subranges therebetween.

Additional excipients for use in dosage forms or drug carrier cores of the invention include, but are not limited to, binders, lubricants, glidants, disintegrants, diluents, coloring agents, suspension agents or flavoring agents, and the same excipient may be used for more than one function in a given formulation. Such excipients are described in Remington, The Science and Practice of Pharmacy, 22nd Ed. 2013, which is incorporated herein by reference in its entirety.

Other commonly used pharmaceutically acceptable excipients which may be suitable for use in the present invention include, but are not limited to, water, magnesium stearate, starch, lactose, microcrystalline cellulose, stearic acid, sucrose, talc, silicon dioxide, gelatin, acacia and dibasic calcium phosphate (Baldrick, P. (2000) Regul. Toxicol. Pharmacol. October 32 (2): 210; incorporated herein by reference.) Excipients are combined with active ingredients for example to enhance appearance, improve stability, aid processing or aid disintegration after administration, but many other excipient functions are known in the art that can be applied to oral dosage forms of the present invention. Classes of excipients which are often used and suitable for use in the present invention include but are not limited to, natural, modified-natural or synthetic mono-, oligo- or polysaccharides where oligo- and polysaccharides may or may not be physically or chemically crosslinked; natural, modified-natural or synthetic mono-, oligo- and polypeptides or proteins where oligo- and polypeptides and proteins may or may not be physically or chemically crosslinked; synthetic oligomers and polymers that may or may not be physically or chemically crosslinked; monomeric, hydrophobic, hydrophilic or amphoteric organic molecules; inorganic salts or metals; and combinations thereof. Accordingly, therapeutic agents used herein such as, for example, GHB, paracetamol, codeine or oxycodone may be combined with any excipient(s) known in the art that allows tailoring its performance during manufacturing, administration and/or its in vitro and in vivo performance.

Material which helps to hold the bulk of a product together and/or helps to maintain the product in a desired shape is known as a “binder” or “granulator”. Binders suitable for use in the present invention are exemplified by, but are not limited to, sugars, gelatin, gums, microcrystalline cellulose and other modified celluloses, waxes or synthetic polymers like polyethylene glycol or polyvinyl pyrrolidone. Additional excipients often utilized in product formulations are lubricants. These are substances which aid in the manufacturing process as they help minimize clumping of the products and also help release them from the manufacturing machinery. A common “lubricant” used for pharmaceutical formulations is magnesium stearate; however, other commonly used product lubricants include talc, calcium stearate, stearic acid (stearin), hydrogenated vegetable oils, sodium benzoate, leucine, carbowax 4000 and sodium stearyl fumarate all of which may be suitable for use in the present invention. Glidants also referred to as “flow-aids”, help to keep the powder or dry material of the products flowing as the products are being made, stopping them from forming lumps. Examples of commonly used glidants which may be suitable for use in the invention include colloidal silicon dioxide, talc, calcium silicate and magnesium silicate. Disintegrants are often added to pharmaceutical formulations to induce breakup of the product or dosage form (i.e. pellet or tablet) when it comes in contact with aqueous fluid in order to help release the drug. The objectives behind addition of disintegrants are to increase surface area of the product fragments and to overcome cohesive forces that keep these particles together in a formulation. They do this by promoting wetting and swelling of the dosage form so that it breaks up in the gastrointestinal tract. Some binders such as starch and cellulose also act as disintegrants. Other disintegrants are clays, cellulose derivatives, algins, gums and crosslinked polymers. Another group of disintegrants called “super-disintegrants” may be utilized. These materials are effective at low (2-5%) concentrations. “Super-disintegrants” which may be suitable for use in the present invention include, but are not limited to, sodium starch glycolate (SSG), croscarmellose sodium or crosprovidone.

It could be envisaged that a material or materials which help suspend a composition of the invention in a liquid, for example water, for administration could be used. In some embodiments the formulation comprises a suspension agent or viscosity modifying agentselected from the group consisting of but not limited to acacia, agar, alginic acid, bentonite, calcium stearate, carbomers, carboxymethylcellulose calcium, carboxymethylcellulose sodium, carrageenan, cellulose (powdered), ceratonia, colloidal silicon dioxide, dextrin, gelatin, guar gum, hectorite, hydrophobic colloidal silica, hydroxyethyl cellulose, hydroxyethylmethyl cellulose, hydroxypropyl cellulose, hypromellose, kaolin, magnesium aluminium silicate, maltitol solution, medium-chain triglycerides, methylcellulose, microcrystalline cellulose, phospholipids, polycarbophil, polyethylene glycol, polyoxyethylene sorbitan fatty acid esters, potassium alginate, povidone, propylene glycol alginate, saponite, sesame oil, sodium alginate, sodium starch glycolate, sorbitan esters, sucrose, tragacanth, vitamin E polyethylene glycol succinate and xanthan gum.

In some embodiments, the formulation comprises one or more pH adjusting agents. In embodiments, the pH adjusting agent is an acid, base or a salt. For example, as described in U.S. Pat. No. 8,263,650, which is incorporated by reference for all purposes. In some embodiments, the pH adjusting agent is an acid selected from the group consisting of acetic, acetylsalicylic, barbital, barbituric, benzoic, benzyl penicillin, boric, caffeine, carbonic, citric, dichloroacetic, ethylenediaminetetraacetic acid (EDTA), formic, glycerophosphoric, glycine, lactic, malic, mandelic, monochloroacetic, oxalic, phenobarbital, phenol, picric, propionic, saccharin, salicylic, sodium dihydrogen phosphate, succinic, sulfadiazine, sulfamerazine, sulfapyridine, sulfathiazole, tartaric, trichloroacetic, and the like, or inorganic acids such as hydrochloric, nitric, phosphoric or sulfuric, and the like. Preferably the pH adjusting agent should be a pharmaceutically acceptable acid as listed in the “Handbook of Pharmaceutical Salts: Properties, Selection and Use” (P. Stahl; John Wiley & Sons, Aug. 4, 2008; included herein by reference).

GHB is the preferred therapeutic agent for use in formulations of the invention. Typical concentrations of solid and liquid GHB formulations are shown in U.S. Pat. Nos. 8,263,650 and 8,324,275.

### Methods of the Present Disclosure

In some embodiments, the present invention is a method for treating a GHB-treatable disorder, the method comprising administering at least two forms of GHB to a patient in need thereof, wherein the administration of each form provides a different pharmacokinetic profile and each form is administered on different days. In some embodiments, dose adjustments are important when different release rates are desired to increase or reduce an effect of GHB. In some embodiments, an immediate release (IR) form may be used in conjunction with a modified release (MR) form on different days. For example, one may want to select an IR form on some days to: increase the intended effect (e.g., promote more rapid initiation of sleep at bedtime); decrease unintended effect (e.g. reduce drug-related sleepiness at end of sleep period); to leverage a period of wakefulness associated with a second dosing event or sleep period; or to shift an effect to a different part of a sleep period to increase a therapeutic effect. One may want to select a form having both IR/MR functionality on days other than when the IR form is used to: reduce an unintended effect (e.g., reduce effect of rapid initiation of sleep at bedtime); increase an intended effect (e.g. facilitate “sleeping in”) to obviate period of wakefulness associated with second dosing event or sleep period; or to shift an effect to a different part of a sleep period to increase a therapeutic effect. Other dose forms can be administered to achieve the above goals.

In some embodiments, the present invention provides a method to adjust GHB dosing in which different forms are provided to a patient on different days. This need can be created by work schedules, personal events, or other motivations.

In embodiments, a health care provider (HCP) co-prescribes a custom dosing regimen or a preset dosing regimen, having a defined dosing period, to adjust the timing of wakefulness, such as on a workday versus a non-workday. For example, the patient can be administered an IR form from Sunday night to Thursday night and an IR/MR form on Friday and Saturday nights. There may also be a desire to incorporate second nightly dose (breaking up the once nightly dose into two nightly doses by administering an IR form) on select nights. For example—patient has late dinner every Monday and desires the rising for second nightly dose to urinate, as their fluid intake is always shifted later on Monday nights. Also, there may be a desire to shift to IR/MR dosing for every night of week-long vacation, as precision of wakefulness may not be critical.

In some embodiments, the patient is treated for one or more conditions treatable by administration of GHB. For example, a condition selected from one or more sleep related disorders such as, e.g., narcolepsy (including cataplexy), excessive daytime sleepiness, idiopathic hypersomnia, cataplexy, sleep paralysis, apnea, narcolepsy sleep time disturbances, hypnagogic hallucinations, sleep arousal, insomnia, and nocturnal myoclonus. In some embodiments, the patient is treated for disorders related to drug abuse, alcohol and opiate withdrawal, a reduced level of growth hormone, anxiety, analgesia, effects in certain neurological disorders such as Parkinson's Disease, depression, certain endocrine disturbances and tissue protection following hypoxia/anoxia such as in stroke or myocardial infarction, or for an increased level of intracranial pressure or the like. See U.S. Pat. Nos. 6,472,431; 6,780,889; 7,262,219; 8,263,650; 8,461,203, 8,591,922, 8,901,173, 9,132,107, 9,555,017, 9,795,567, 10,195,168, U.S. Ser. Nos. 16/688,797, and U.S. Patent Publication No. 2018/0263936 for example.

Prior art methods currently administer a daily, therapeutic dose for a patient based on a titration schedule to achieve an effective dose for current formulations. However, some embodiments of the present invention is a method for administering different forms of GHB to a patient in need thereof that is customized for different days. For example, the dosing for a patient could use one GHB form for one day or series of days and another form for a different day or series of days. One form could be administered for 1, 2, 3, 4, 5, 6, 7, 8, 9, 10 or more days in a row and a second form could be administered for a similar period, such as 1, 2, 3, 4, 5, 6, 7, 8, 9, 10 or more days in a row. Also, different forms could be administered alternatively for 1, 2, 3, 4, 5, 6, 7, 8, 9, 10 or more days. In one embodiment of the invention there is an alternating administration of different GHB forms over one dosing period, i.e. the time for which a HCP writes the prescription. Other variables include the number of doses administered during a sleep period, the division of total nightly dose administered during a sleep period, and the changes in the division of total nightly dose administered during a sleep period.

In some embodiments, the GHB form is administered to produce heightened alertness, to modify the administration of GHB, including the form and the number of nightly doses, to increase the intended effect and/or reduce unintended effects for a given patient; i.e., optimize/customize/adapt the administration. The goal is to modify the administration of drug to benefit the patient, including the form and the number of nightly doses.

The different forms of GHB could include the following, singly or in combination over multiple days: immediate release, modified release, continuous release, sustained release, mixed salt, alcohol rugged, or any other forms that are or could be made available and that have different GHB release properties that one could exploit. One embodiment of the invention includes the administration of a mixed salt, immediate release form of GHB (such as Xywav®) on specific days, and on other days, the administration of a form capable of being taken once nightly, which can include immediate release and sustained release forms, such as that shown in U.S. Pat. No. 10,758,488 or U.S. Ser. No. 16/688,797. The immediate release forms of GHB, like Xyrem® and Xywav® are typically taken more than once per night for most people. However, each form could be prescribed in some instances once, twice or thrice per night. Anticipated use of alcohol can create a need to substitute an alcohol rugged form (see U.S. patent application Ser. No. 16/688,797).

The present invention is also a drug distribution system in which at least two forms of a GHB formulation are provided to a patient to treat a sleep related disorder, comprising co-prescribing the two forms to be delivered within the same dosing period, the two forms are to be administered on different days in the same dosing period. The two forms include an immediate release and a modified release form. In some embodiments, the invention is a method for treating a GHB-treatable disorder, the method comprising: administering at least two forms of GHB to a patient in need thereof, wherein the administration of each form provides a different pharmacokinetic profile and each form is administered on different days, wherein each form is administered within 10 days of each other. In some embodiments, the present invention also includes a package of doses of GHB including unit dose forms comprising at least two forms of GHB having different release rates and/or administration instructions, the at least two forms having an immediate release form and a modified release form to be administered on different days, wherein the immediate release form is administered, once, twice or three times daily and the modified release form is administered once daily.

In some embodiments, the formulation has less than 50%, 40%, 30%, 20%, 10% sodium as a cation. In some embodiments, the formulation has less than about 8% sodium as a cation. One preferred form that is commercially available is Xywav®. (See U.S. Pat. No. 8,591,922).

The dosing period could be prescriber-oriented, e.g. a prescription of doses for a period of time (e.g. 1 month); or pharmacy-oriented (e.g. a patient supply of doses to be used over a period of time); or based any other criteria. The appropriate packaging could include all the doses needed for each of these groupings for the dosing period.

One example of a simple dosing schedule could include one that would fit a normal workweek. For example, a patient could take one form, such as Xywav, Sunday through Thursday and a modified release form on Friday and Saturday. Further customization can be performed for intervening periods of vacation, school terms, or other schedule changes.

A customized dosing schedule can be prescribed by a health care practioner (HCP), filled by a pharmacist, and tracked by a Risk Evaluation and Mitigation System (REMS, see U.S. Pat. No. 8,731,963 and 63/199,481 among others). The dose forms can be provided to the patient through a REMS and in packaging that fulfills the patient needs for a specific period of time, such as a dosing period of a week, month, multiple months or in other time increments. The multiple, different GHB dose forms can be packaged separately or together in pre-packaged or unit dose packaging, such as blister packs. The unit dose packages are premeasured (optionally in sachets), protect the medication, and can be bar coded for efficient distribution and tracking. The doses can be packaged at the pharmacy or at the manufacturer and then later assembled at the pharmacy into different doses to be taken during the dosing period. The REMS will track the HCP, patient, and the delivery of the doses according to the process described in U.S. Pat. No. 8,731,963 and U.S. Ser. Nos. 63/199,481, 17/209,608, 17/061,783, among others. Of course, last minute schedule changes that necessitate a change in the preset dosing schedule are contemplated by the present invention (e.g. overnight return and replacement of forms to accommodate the new preset schedule).

The dosing period can be any period for which an HCP prescribes the medication. It could be up to 1, 2, 3, 4, 5, 6, 7, 8, 9, 10, 11, 12 or more weeks, or up to 1, 2, 3, 4, 5, 6, 7, 8, 9 or more months. During that time more than one GHB form is prescribed for different days on a recurring basis and the different doses for that period, or a subset, may be delivered. For example, an HCP could prepare a custom prescription, provide it to the pharmacy and they can prepare to deliver more than one GHB form in a single delivery for the dosing period. This arrangement can continue until the prescription is modified or otherwise discontinued.

### Methods of Making

The mixed salt oxybate, compositions and formulations may be prepared using methods that are known to those skilled in the art, including the methods described U.S. Pat. Nos. 8,591,922; 8,901,173; 9,132,107; 9,555,017; 10,195,168 and U.S. Publication No. 2018/0263936, which are hereby incorporated by reference.

All, documents, patents, patent applications, publications, product descriptions, and protocols which are cited throughout this application are incorporated herein by reference in their entireties for all purposes.

What is claimed:

1. A method for treating a GHB-treatable disorder, the method comprising:
administering at least two forms of GHB to a patient in need thereof, wherein the administration of each form provides a different pharmacokinetic profile and each form is administered on different days.

2. The method of claim 1, wherein the forms of GHB administered to the patient are selected to modify different effects.

3. A method for treating a GHB-treatable disorder comprising:
administering at least two forms of GHB having different GHB release rates to a patient in need thereof, wherein on one day, or series of days, an immediate release form is administered to the patient; and wherein a modified release form is administered to the patient on the other days.

4. The method of claim 3, wherein the immediate release form is administered more than once per night and the modified release form is administered once per night.

5. The method of any one of claims 1-4, wherein the GHB-treatable disorders are one or more conditions selected from the group consisting of narcolepsy, cataplexy, excessive daytime sleepiness, idiopathic hypersomnia, cataplexy, sleep paralysis, apnea, narcolepsy sleep time disturbances, hypnagogic hallucinations, sleep arousal, insomnia, and nocturnal myoclonus or disorders related to drug abuse, alcohol and opiate withdrawal, a reduced level of growth hormone, anxiety, analgesia, effects in certain neurological disorders such as Parkinson's Disease, depression, certain endocrine disturbances and tissue protection following hypoxia/anoxia such as in stroke or myocardial infarction, or for an increased level of intracranial pressure or the like.

6. The method of any one of claims 1-5, wherein a health care provider co-prescribes a custom dosing regimen or a preset dosing regimen, having a defined dosing period, under conditions in which one form is administered to achieve one clinical effect and another form is administered to achieve a different clinical effect.

7. A drug distribution system for providing at least two forms of GHB to a patient to treat a sleep related disorder, comprising instructing the patient to administer the two forms within a dosing period, wherein the patient is instructed to administer the two forms on different days in the dosing period.

8. The drug distribution system of claim 7, wherein the two forms comprise an immediate release form and a modified release form.

9. A packaged product containing GHB unit doses, wherein the unit doses comprise at least two forms of GHB having different release rates.

10. The packaged product of claim 9, wherein the at least two forms of GHB comprise an immediate release form and a modified release form.

11. The packaged product of claim 10, wherein the packaged product further comprises written instructions that the patient should administer the immediate release form and the modified release form on different days.

12. The packaged product of claim 11, wherein the written instructions provide that the immediate release form is administered twice daily, and the modified release form is administered once daily.

13. The packaged product of any one of claims 9-12, wherein the unit dose comprises blister packs or sachets.

14. The method of any one of claims 1-6, the distribution system of any one of claims 7-8, or the packaged product of any one of claims 9-13, wherein the GHB is present in a formulation having two or more cations.

15. The method of any one of claims 1-6, the distribution system of any one of claims 7-8, or the packaged product of any one of claims 9-13, wherein the GHB is present in a formulation having two or more cations selected from the group consisting of sodium, potassium, magnesium and calcium.

16. The method of any one of claims 1-6, the distribution system of any one of claims 7-8, or the packaged product of any one of claims 9-13, wherein the formulation has less than 50%, 40%, 30%, 20%, 10% sodium as a cation, preferably 8% sodium.

17. The method of any one of claim 1-6 or 15-17, wherein the GHB form administered is selected to produce heightened alertness, to modify the administration of GHB, including the form and the number of nightly doses, to increase the intended effect and/or reduce unintended effects for a given patient; i.e., optimize/customize/adapt the administration.

18. The method of any one of claim 1-6 or 15-17, wherein the GHB is administered in different forms on different days, such as one form on the nights preceding workdays and a different form on nights preceding non-workdays.

19. The method of any one of claim 1-6 or 15-17, wherein a twice nightly form is administered on Sunday through Thursday nights and a once nightly form is administered on Friday and Saturday nights.

20. The method of any one of claim 1-6 or 15-17, wherein the GHB is administered at a reduced dosage when co-administered with sodium valproate.

21. The method of any one of claim 1-6 or 15-17, wherein the GHB is distributed using a system having a central database and a single pharmacy.

22. The method of any one of claim 1-6 or 15-17, wherein the administration of an immediate release form provides heightened alertness in the wake period following administration compared to the wake period following administration of a sustained release form.
